(19) United States
(12) Patent Application Publication
(10) Pub. No.: US 2014/0224736 A1
(43) Pub. Date: Aug. 14, 2014

(54) DEVICE AND METHOD FOR REGULATING A TREATMENT DEVICE

(71) Applicant: FRESENIUS MEDICAL CARE DEUTSCHLAND GMBH, BAD HOMBURG (DE)

(72) Inventors: Alexander HEIDE, Eppstein (DE); Dejan NIKOLIC, Frankfurt (DE); Arne PETERS, Bad Homburg (DE); Christoph WIKTOR, Gelnhausen (DE)

(73) Assignee: FRESENIUS MEDICAL CARE DEUTSCHLAND GMBH, BAD HOMBURG (DE)

(21) Appl. No.: 14/178,591

(22) Filed: Feb. 12, 2014

Related U.S. Application Data
(60) Provisional application No. 61/764,223, filed on Feb. 13, 2013.

(30) Foreign Application Priority Data
Feb. 13, 2013 (DE) 102013002395.5

(51) Int. Cl. A61M 1/16

(52) U.S. Cl. CPC A61M 1/1601; A61M 2202/0413; A61M 2205/33
USPC 210/646; 210/101; 210/143

(57) ABSTRACT

A method and a device are disclosed for controlling or regulating an ultrafiltration in a dialysis treatment in which blood to be ultrafiltered flows in an extracorporeal blood circulation (109) through a blood chamber (110) of a dialyzer (113), which is divided by a semipermeable membrane (111) into the blood chamber (110) and a dialysis fluid chamber (108), and dialysis fluid in a dialysis fluid circulation (109) flows through the dialysis fluid chamber (108) of the dialyzer (113). The device has a blood pump (115) for creating a blood flow in the extracorporeal blood circulation (112), a dialysis fluid pump (107) for creating a dialysis fluid flow in the dialysis fluid circulation (109), a balancing device (104) for setting up a fluid balance in the dialysis fluid circulation between an inflow (106) and an outflow (105) of the dialysis fluid chamber (113) as a measure of the ultrafiltration, and a regulating unit (101) for regulating the blood pump (115) and/or the dialysis fluid pump (107). The regulation is performed in such a way that a predetermined ultrafiltration is achieved without any further active control or regulation of the dialysis fluid flow flowing into the dialyzer (113) or flowing out of the dialyzer (113).

### TECHNICAL FIELD

The present invention relates to a method and a device for regulating a treatment device, in particular for regulating the ultrafiltration in a dialysis treatment.

### BACKGROUND

Dialysis is a method for purifying the blood of patients with acute or chronic renal failure. A fundamental distinction is made here between methods using an extracorporeal blood circulation such as hemodialysis, hemofiltration or hemodiafiltration, and peritoneal dialysis which does not have an extracorporeal blood circulation.

In hemodialysis blood is passed in an extracorporeal circulation through the blood chambers of a dialyzer, which is separated from a dialysis fluid chamber by a semipermeable membrane. A dialysis fluid containing the blood electrolytes in a certain concentration flows through the dialysis fluid chamber. The substance concentration of the blood electrolytes in the dialysis fluid corresponds to the concentration in the blood of a healthy subject. During the treatment, the patient's blood and the dialysis fluid are passed by both sides of the semipermeable membrane, usually in countercurrent at a predetermined flow rate. Substances that are eliminated in urine will diffuse through the membrane from the blood chamber into the chamber for dialysis fluid, while at the same time electrolytes present in the blood and in the dialysis fluid will diffuse from the chamber of the higher concentration to the chamber of the lower concentration. If a pressure gradient builds up from the blood side to the dialysate side on the dialysis membrane, water will go from the patient's blood through the dialysis membrane and into the dialysis circulation to form the so-called ultrafiltrate. This process of ultrafiltration leads to the desired withdrawal of water from the patient's blood.

In hemofiltration, ultrafiltrate is withdrawn from the patient's blood by applying a transmembrane pressure in the dialyzer without passing the dialysis fluid over the membrane of the dialyzer on the side opposite the patient's blood. In addition, a sterile and pyrogen-free substituate solution may be added to the patient's blood. Depending on whether this substituate solution is added upstream from the dialyzer or downstream, we speak of predilution or postdilution. The mass exchange in hemofiltration takes place by convection.

It is a combination of hemodialysis and hemofiltration when substituate is added to the patient's blood at the same time in a dialysis treatment. This form of treatment, which should also be referred to as hemodiafiltration, is also covered in the following discussion by the term hemodialysis, dialysis or dialysis treatment.

In a dialysis treatment it is of crucial importance for the withdrawal of fluid to be measured and balanced with a high precision because even a slightly excessive withdrawal of fluid could have serious consequences for the patient.

This is ensured by the fact that the inflow of dialysate or dialysis fluid into the dialysis fluid chamber and the outflow of dialysis fluid out of the dialysis fluid chamber are controlled and monitored separately from one another. The balance between the quantity of fluid supplied to the dialysis fluid chamber and the quantity of fluid removed from the dialysis fluid chamber at the same time yields a measure for the quantity of ultrafiltrate taken from the patient's blood.

One possibility for balancing is the use of balancing chambers, which are based on the principle that a quantity of fluid supplied in an inflow to the dialysis fluid chamber corresponds to a quantity of fluid removed in an outflow out of the dialysis fluid chamber.

For the additional withdrawal of fluid from the patient, another flow path with a delivery device, the so-called ultrafiltration pump, is arranged in parallel with the balancing chamber. The quantity of fluid to be withdrawn is measured by the parallel flow path past the balancing chamber and through the ultrafiltration pump and thus forms a measure for the fluid balance.

Balance chambers have a complex structure and make high demands of the manufacturing tolerance.

Alternatively, the control of ultrafiltration may be controlled and monitored by controlling the flow rate in the inlet line to the dialysis fluid chamber and the flow rate in the outlet line out of the dialysis fluid chamber through independently controllable pumps arranged in the inlet line and in the outlet line. In this case, the balancing is performed by the respective flow sensors or scales arranged in the inlet line and in the outlet line, but this is associated with a great expense and complexity for the calibration of these sensors or scales.

Therefore one object of the present invention is to overcome at least one of the aforementioned difficulties and to make available a simple device and a corresponding method for regulating ultrafiltration.

### SUMMARY

This object is achieved by a device for regulating an ultrafiltration in a dialysis treatment in which blood to be ultrafiltered flows in an extracorporeal circulation through a blood chamber of a dialyzer, which is subdivided by a semipermeable membrane into the blood chamber and a dialysis fluid chamber, and dialysis fluid flows through the dialysis fluid chamber of the dialyzer in a dialysis fluid circulation. The device according to the disclosure has a blood pump for creating a blood flow in the extracorporeal blood circulation, a dialysis fluid pump for creating a dialysis fluid flow in the dialysis fluid circulation, a balancing device for setting up a fluid balance in the dialysis fluid circulation between an inflow and an outflow out of the dialysis fluid chamber as a measure of the ultrafiltration as well as a regulating unit for regulating the blood pump and/or the dialysis fluid pump. The blood pump and/or the dialysis fluid pump is/are regulated, so that a predetermined ultrafiltration is achieved without additional active control or regulation of the dialysis fluid flow coming into the dialyzer or leaving the dialyzer.

In addition, the object of the present invention is achieved by a device according to Claim 1 as well as by a method according to Claim 11 for regulating an ultrafiltration in a dialysis treatment. Advantageous embodiments are defined in the Dependent Claims.

The dialysis fluid pump is advantageously arranged in an inlet line to the dialysis fluid chamber. In this way, the dialysis fluid pump may be arranged close to a dialysis fluid preparation site.

The inventors have recognized that with this configuration, no additional active control or regulation of the dialysis fluid flow downstream from the dialysis fluid chamber is necessary. This is associated with a reduced structural complexity.

Regulation of ultrafiltration, in particular the ultrafiltration rate or the ultrafiltration volume may in this case be accomplished exclusively through the control and/or regulation of the blood pump and of the dialysis fluid pump.

Additionally advantageously the blood pump is arranged in the blood circulation in an inlet line to the blood chamber. In this way the pressure supplied by the blood pump contributes toward an excess pressure in the blood chamber in comparison with the dialysis fluid chamber.

The ultrafiltration may be regulated so that a predetermined value can be set for the pressure applied by the blood pump and the regulating unit regulates the ultrafiltration by regulating the dialysis fluid pump as a function of a measured ultrafiltration rate, for example, by regulating a pressure or a volume flow in an inlet line to the dialysis fluid chamber in the dialysis fluid circulation accordingly.

Alternatively the regulation of the ultrafiltration may also take place in such a way that a predetermined value can be set for the dialysis fluid rate or for the delivery pressure of the dialysis fluid pump, and the regulating unit regulates the ultrafiltration by regulating the blood pump as a function of a measured ultrafiltration rate in that the pressure or the volume flow in an inlet line to the blood chamber in the extracorporeal blood circulation is regulated accordingly.

However any other regulation of ultrafiltration is also possible as long as the pressure conditions on the dialyzer can be controlled and/or regulated through the blood pump in the blood circulation and through the dialysis fluid pump in the dialysis fluid circulation so that the desired ultrafiltration is achieved.

In a further embodiment of the device, the balancing device has a differential flow-measuring unit for measuring the differential flow between a flow in the inlet flow to the dialysis fluid chamber and the outlet flow out of the dialysis fluid chamber, a branch from the inlet flow or the outlet flow to the branch of the dialysis fluid from the inlet flow or the outlet flow into another flow path as well as a device for adjusting the flow rate in the inlet flow, the outlet flow and/or in the additional flow path which can be controlled in such a way that the measured differential flow satisfies a predetermined condition. In this further embodiment the device also has a unit for determining the flow rate in the additional flow path as a measure of the fluid balance.

The flow rate in the inflow or the outflow out of the dialysis fluid chamber may be adjusted, for example, with the help of a dialysis fluid pump arranged in the inflow or in the outflow. The flow in the additional flow path can be adjusted with a pump arranged in the flow path.

### BRIEF DESCRIPTION OF THE FIGURES

FIG. 1 shows a block diagram of a dialysis machine with a device for regulating the ultrafiltration.

FIG. 2 shows a block diagram of another dialysis machine with another device for regulating the ultrafiltration.

FIG. 3 shows a block diagram of an equivalent circuit diagram for a dialysis machine.

FIG. 4 shows a block diagram of a simplified electric circuit diagram for a dialysis machine.

### DETAILED DESCRIPTION OF THE FIGURES

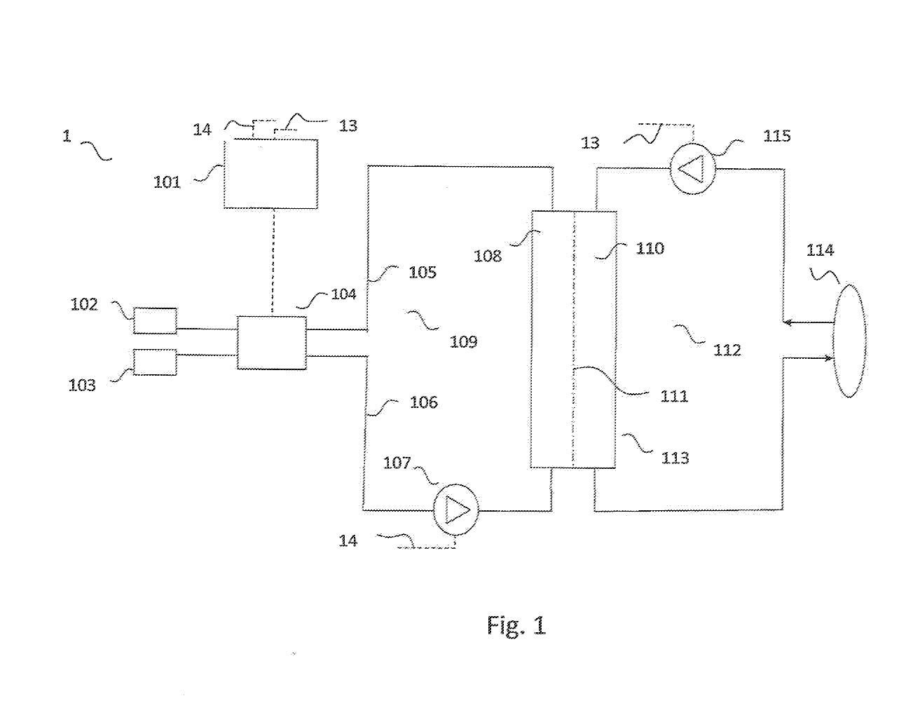

FIG. 1 shows schematically a dialysis machine 1 with a device for regulating ultrafiltration in accordance with the teaching of the present invention. The blood to be treated is taken from, the patient via an access 114 and then is returned back to the patient with a blood pump 115 in the extracorporeal blood circulation 112 through a blood chamber of the dialyzer 113 and through the access 114. The access 114 connects the blood circulation 112 to a blood vessel of the patient which is suitable for withdrawing blood and returning blood. The access 114 may contain a separate inlet and outlet for withdrawing blood and for returning blood (“double needle” method) or the inlet and outlet may be embodied as a single element (“single needle” method).

In the dialyzer 113 a semipermeable membrane 111 separates a dialysis fluid chamber 108 from a blood chamber 110. There is a fluid and mass exchange from the blood chamber 110 into the dialysis fluid chamber 108 by way of the semipermeable membrane 111. Dialysis fluid is conveyed through the dialysis fluid chamber 108 of the filter 113 in the dialysis fluid circulation 109 by means of a dialysis fluid pump 107 in an inlet line 106 upstream from the dialysis fluid chamber. Alternatively the dialysis fluid pump may also be arranged in an outlet line 105 downstream from the dialysis fluid chamber. A balancing device 104 for balancing the dialysis fluid supplied to the dialysis fluid chamber and flowing out of the dialyzer is arranged in the dialysis fluid circulation 109, which is fed from a dialysis fluid source 103. To do so, the flow rate in the inflow to the dialysis fluid chamber and the flow rate in the outflow from the dialysis fluid chamber can be detected separately or a differential flow may be determined as a measure of the fluid balance. The fluid balance corresponds to the ultrafiltration quantity withdrawn through the membrane in the dialyzer. So-called spent dialysis fluid flowing out of the dialyzer is usually discarded in a dialysis fluid drain 102. Alternatively, regeneration of spent dialysis fluid may be provided.

The pressure conditions on the membrane 111 in the dialyzer 113 are influenced by the control of the blood pump 115 and the control of the dialysis fluid pump 107 so that an excess pressure prevails in the blood chamber 110 in comparison with the dialysis fluid chamber 108. Therefore fluid is transported through the membrane from the blood chamber 110 into the dialysis fluid chamber 108.

The blood pump may be controllable to achieve a certain pump rotational speed or a certain blood flow rate as an operating parameter, for example, in one embodiment as a peristaltic pump. Alternatively, the blood pump may also be controllable to achieve a certain delivery pressure as an operating parameter, for example, controlling the pump as an impeller pump.

Likewise, to achieve a certain delivery rate or pump rotational speed, the dialysis fluid pump may be designed as a peristaltic pump, a diaphragm pump, a piston pump or the like or for establishing a certain delivery pressure, e.g., as an impeller pump.

A control and regulating unit 101 connected to the balancing device 104 via a measurement line is connected to the blood pump 115 via the control line 13 and to the dialysis fluid pump 107 via the control line 14. During the blood treatment, current measurement parameters of ultrafiltration, for example, the ultrafiltration quantity or ultrafiltration rate, are transferred either periodically or continuously from the balancing device to the control and regulating unit 101. The control and regulating unit 101 uses the current measurement parameters to derive control signals for the blood pump 115 and for the dialysis fluid pump 107. The dialysis fluid pump 107 and the blood pump 115 are controlled with respect to an ultrafiltration that is to be achieved, for example, a certain ultrafiltration rate or a certain ultrafiltration volume to be achieved over the course of treatment.

The regulation may be implemented in such a way that the blood pump 115 is operated at a constant rotational speed or at a constant delivery pressure and the dialysis fluid pump 107 is controlled, so that the ultrafiltration value transmitted by the balancing device 104 serves as a manipulated variable. For example, if the value for the ultrafiltration rate thus transmitted is above a corresponding ideal value, then the dialysis fluid pump 107 is to be accelerated but if the ultrafiltration rate is below its ideal value, the dialysis fluid pump 107 is throttled.

One alternative control strategy may be to operate the dialysis fluid pump 107 at a constant rotational speed or at a constant delivery pressure and to control the blood pump 115, so that the ultrafiltration value transmitted by the balancing device 104 is regulated. For example, if the value for the ultrafiltration rate thus transmitted is above a corresponding ideal value, then the blood pump 115 should be throttled and if the ultrafiltration rate is below its ideal value then the blood pump 115 is accelerated.

A combination of control strategies is possible, for example, by first operating the blood pump 115 at a constant rate in an internal control loop and controlling the dialysis fluid pump 107. Only when a limit value has been reached for the dialysis fluid pump is the blood pump 115 controlled accordingly.

An alternative combination would consist of operating the dialysis fluid pump at a constant rate in a type of internal control loop and controlling the blood pump 115. When a limit value for an operating parameter of the blood pump 115 has been reached, the dialysis fluid pump is then also controlled.

Regulation of ultrafiltration may be accomplished by stipulating a certain value for the ultrafiltration rate. Alternatively, a certain ultrafiltration profile may be stipulated for the ultrafiltration volume to be withdrawn during the blood treatment.

The stipulated value of the ultrafiltration rate may be a constant value or a continuously varying value for the ultrafiltration rate.

Alternatively, a profile in which intervals with a positive ultrafiltration rate alternate with intervals with a negative ultrafiltration rate may be stipulated for the ultrafiltration rate or the ultrafiltration volume. A so-called push-pull mode, in which deposits are released from the dialyzer membrane or deposits of substance on the dialyzer membrane are reduced or prevented, can be achieved in this way. The permeability of the dialyzer membrane and the corresponding cleaning performance (clearance) for medium-sized molecules is improved in this way. The present arrangement achieves this without any additional equipment expense, for example, without an additional pump for creating the oscillating pressure pulses.

The regulation of ultrafiltration may be accomplished in this case by methods like the control strategies described above for the ultrafiltration rate such that instead of a balancing with the ideal value of the ultrafiltration rate, a corresponding comparison with an ultrafiltration profile is performed.

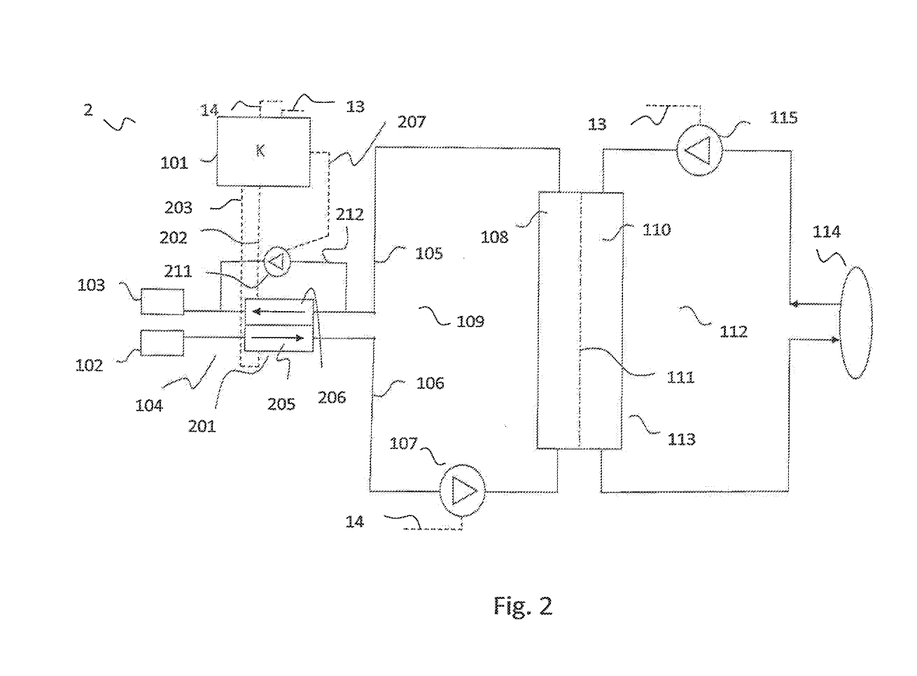

FIG. 2 shows schematically another dialysis machine which another device for regulating ultrafiltration. The dialysis machine shown in FIG. 2 corresponds essentially to the design of the ultrafiltration device in FIG. 1. Reference is made to the description of the corresponding elements instead of repeating the description here. The representation of the ultrafiltration device differs essentially in the design of the balancing device 104, which is described in greater detail below.

The balancing device 104 comprises the flow-measuring cell 205 and 206 which are connected to a differential flow sensor 201, such that the flow-measuring cell 205 is situated upstream from the dialysis fluid chamber 108 and the flow-measuring cell 206 is situated downstream from the dialysis fluid chamber 108 in the dialysis fluid circulation 109.

An ultrafiltration pump 211 is situated in a fluid path 212, which is parallel to the fluid-measuring cell 206, the fluid transport being controlled through the ultrafiltration pump 211.

The differential flow sensor 201 determines a pair of measured values consisting of a separate measured value for each flow-measuring cell 205, 206, which indicates the velocity of flow of the fluid through the respective flow-measuring cell. The pair of measured values is preferably determined one or more times per second and transmitted to the control and regulating unit 101 via measuring lines 202 and 203. The control and regulating unit 101 assigns a volume flow pair to each measured value pair, so that mapping of a measured value onto a volume flow can be used, this image being based on a calibration performed previously. Alternatively, there could also be mapping onto a mass flow. The control and regulating unit 101 derives a control signal for the pump 211 from the volume flow pair thus determined, for example, the pump 211 being operated so that the volume flow through both flow-measuring cells 205 and 206 of the differential flow sensor is the same. For example, the control and regulating unit 101 forms a differential signal from the two volume flows of the volume flow pair and adjusts the flow rate of the ultrafiltration pump 211 by increasing or reducing it depending on the plus or minus sign of the differential signal in a suitable manner, so that the differential signal becomes negligible. If the flow through the flow-measuring cell 205 is less than the flow through the flow-measuring cell 206, then a positive value is obtained for the difference between the measured values for the flow-measuring cell 206 and the flow-measuring cell 205. The control and regulating unit 101 may then vary the control signal for the ultrafiltration pump 211, so that the flow rate through the ultrafiltration pump 211 is increased and the flow through the flow-measuring cell 206 is reduced while the flow in the outflow from the dialyzer remains unchanged, until the same flow is established as that through the flow-measuring cell 205. The flow rate through the ultrafiltration pump 211 then indicates the differential flow between the dialysis fluid flow coming out of the dialysis fluid chamber and the dialysis fluid flow entering the dialysis fluid chamber. The flow rate through the ultrafiltration pump 211 is than a measure of the amount of ultrafiltrate withdrawn in the dialyzer 113.

In one embodiment the flow rate is set at a predetermined value through the ultrafiltration pump 211, and the blood pump 115 and the dialysis fluid pump 107 are controlled as described above, so that the differential flow measured in the differential flow sensor 201 fulfills a predetermined condition, for example: becoming negligible.

The flow rate through the ultrafiltration pump 211 here is a measure of the fluid balance between the inflow to the dialyzer 113 and the outflow out of the dialyzer 113, i.e., for the amount of ultrafiltrate withdrawn in the dialyzer 113.

The balancing unit 104 as a whole acts like a passive component and does not cause any active control or regulation of the fluid balance between the dialysis fluid flowing into the dialyzer 113 and that flowing out of the dialyzer.

The disappearance of the differential signal may relate to a differential flow at a certain point in time or to the disappearance of the integral of a differential flow.

In another embodiment the allocation of the measured value pair to a volume flow or mass flow may be omitted if the difference in the measured values at the same volume flow through both channels is known. The control and regulating unit 101 in this case forms the difference of the two measured values and alters the flow rate of the ultrafiltration pump 211 by increasing or reducing the difference in a suitable manner until the difference corresponds to the previously known difference at the same volume flow.

The differential flow sensor 201 may advantageously function according to the magnetic inductive principle in which the two flow-measuring cells 205, 206 through which the fluid flows in countercurrent, have a preferably rectangular cross section and are arranged at a right angle to a magnetic field. The magnetic field is adjusted by the control of the differential current sensor 201 and is such that a homogeneous field prevails through both flow-measuring cells 205, 206. This achieved, for example, by the fact that the channels of the flow-measuring cells 205, 206 are arranged next to one another in a magnetic field. In each channel, an electrode is mounted opposite and at a right angle to the magnetic field and to the direction of flow in the respective channel on the inner channel wall which extends along the magnetic field. If fluid flows through the channel, then a charge separation of the ions present in the fluid is achieved through the magnetic field so that an electric voltage prevails on the electrodes. This voltage is proportional to the velocity of flow and depends on the magnetic field strength. If the magnetic field is the same in both flow-measuring cells 205 and 206, then the magnetic field strength dependence for the relative differential flow signal is advantageously eliminated in forming a differential signal from the two channels.

In other words, disappearance of the differential signal indicates that the flow through the flow-measuring cell 205 and the flow through the flow-measuring cell 206 are equal in size regardless of the absolute size of the magnetic field in the flow-measuring cells 205 and 206.

In the embodiment in which a profile is predefined for the ultrafiltration rate or for the ultrafiltration volume, such that intervals with a positive ultrafiltration rate alternate with intervals with a negative ultrafiltration rate, the predetermined condition is advantageously satisfied when the integral of the ultrafiltration rate and/or the differential signal becomes negligible.

The ultrafiltration pump 211 is preferably selected from the group of displacement pumps, preferably a diaphragm pump, a hose roller pump, a piston pump, a geared pump or any other type of pump that makes it possible to determine the quantity of fluid delivered. For example, the volume delivered with the hose roller pump can be determined with good accuracy based on the pump hose volume and the angle of rotation of the rotor of the hose roller pump using known methods. Corresponding methods are also known from the prior art for other pumps from the group of displacement pumps for determining the quantity of fluid delivered.

It is advantageous here that the quantity of fluid to be measured corresponds to the quantity of ultrafiltrate. This quantity is typically 3-5 liters per dialysis treatment or per day, whereas the quantity of dialysate flowing through the flow sensor typically amounts to 60-240 liters thereof. It is therefore advantageously possible in accordance with the present disclosure to use measuring devices or measurement methods for the differential flow which must have a much lower tolerance than the measurement methods, which detect the quantity of dialysate flowing out and the quantity flowing in and only then form the difference.

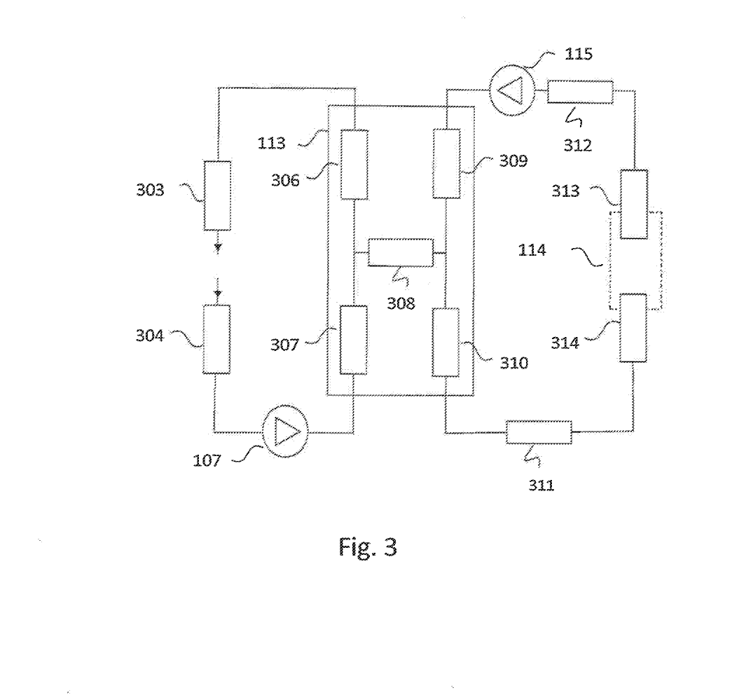

FIG. 3 shows an equivalent diagram of the dialysis machine shown in FIG. 1 with the dialysis fluid pump 107, the blood pump 115 and the dialyzer 113, as well as the flow resistances in the dialysis fluid circulation, in the blood circulation and in the dialyzer are shown as resistances in an electric circuit diagram. Specifically an arterial needle resistance 313, an arterial line resistance 312, a venous needle resistance 314, a venous line resistance 311 are shown in the extracorporeal blood circulation and in the dialyzer 113 this shows an arterial filter longitudinal resistance 309 and a venous filter longitudinal resistance 310. The flow resistances in the dialysis fluid circulation are modeled through an input-end flow resistance of the dialyzer 307, an output-end flow resistance of the dialyzer 306, a flow resistance 304 on the dialysate inlet side of the dialysis fluid circulation and a flow resistance 303 on the dialysate output side of the dialysis fluid circulation. The membrane in the dialyzer is modeled by a transmembrane resistance 308.

TABLE 1

| | Reference | Formula |
|---|---|---|
| Identification of the resistance | numeral | symbol |
| arterial needle resistance in the extracorporeal | 313 | R_{aN} |
| blood circulation | | |
| arterial line resistance in the extracorporeal blood | 312 | R_{aL} |
| circulation | | |
| venous needle resistance in the extracorporeal | 314 | R_{vN} |
| blood circulation | | |
| venous line resistance in the extracorporeal blood | 311 | R_{aN} |
| circulation | | |
| arterial filter longitudinal resistance | 309 | R_{aF} |
| venous filter longitudinal resistance | 310 | R_{vF} |
| input end flow resistance in the dialysis fluid | 304 | R_{Din} |
| circulation | | |
| output end flow resistance in the dialysis fluid | 303 | R_{Dout} |
| circulation | | |
| input end flow resistance of the dialyzer | 307 | R_{DFin} |
| output end flow resistance of the dialyzer | 306 | R_{DFout} |
| transmembrane resistance | 308 | R_{TM} |

Table 1 shows the designations for the individual resistances, their reference numerals and the formula symbols used in the derivation for the derivation given below for dimensioning of the resistances in the dialysis fluid circulation, in the extracorporeal blood circulation and the transmembrane resistance.

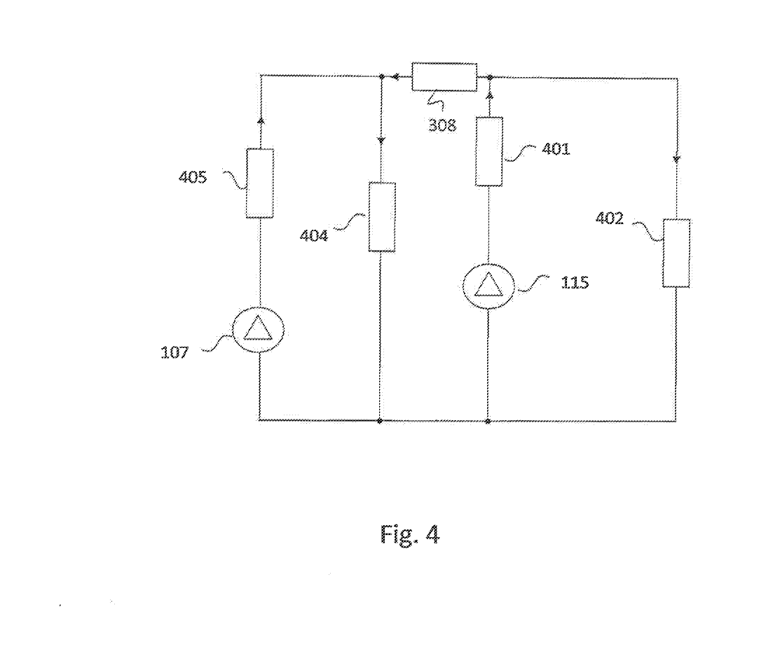

FIG. 4 shows a simplified equivalent circuit diagram of the electric equivalent circuit diagram shown in FIG. 3. In the simplified equivalent circuit diagram shown in FIG. 4, the arterial needle resistance in the extracorporeal blood circulation (symbol: R_{aN}), the arterial line resistance in the extracorporeal blood circulation (symbol: R_{aL}) and the arterial filter longitudinal resistance (symbol: R_{aF}) are combined into a total arterial resistance 401 (symbol: Rₐ) as follows:

Rₐ=R_{aN}+R_{aL}+R_{aF} (equation 1)

Likewise, the venous needle resistance in the extracorporeal blood circulation (symbol: R_{vN}), the venous line resistance (symbol: R_{vL}) in the extracorporeal blood circulation and the venous filter longitudinal resistance (symbol: R_{vF}) are combined into a total venous resistance 402 (symbol: Rᵥ) as follows:

Rᵥ=R_{vN}+R_{vL}+R_{vF} (equation 2)

A corresponding combination of resistances in the dialysis fluid circulation yields the following result. The flow resistance on the input end of the dialysis fluid circulation 304 (symbol: R_{Din}) and the flow resistance 307 of the dialyzer on the input end (symbol: R_{DFin}) may be combined into an input resistance 405 (symbol: Rᵢₙ):

Rᵢₙ=R_{Din}+R_{DFin} (equation 3)

The flow resistance at the output end of the dialysis fluid circulation 303 (symbol: R_{Dout}) and the flow resistance of the dialyzer 306 at the output end (symbol: R_{DFout}) may be combined into an output resistance 404 (symbol: Rₒᵤₜ) as follows:

Rₒᵤₜ=R_{Dout}+R_{DFout} (equation 4).

The designations of the resistances shown in FIG. 4, their reference numerals and the symbols used in the derivation are shown in Table 2 below.

TABLE 2

| | Reference | Formula |
|---|---|---|
| Identification of the resistance | numeral | symbol |
| total arterial resistance | 401 | Rₐ |
| total venous resistance | 402 | Rᵥ |
| input resistance (in the dialysis fluid circulation) | 405 | Rᵢₙ |
| output resistance (in the dialysis fluid circulation) | 404 | Rₒᵤₜ |
| transmembrane resistance | 308 | R_{TM} |

The blood pump and the dialysis fluid pump may be modeled as a current source or as a voltage source, wherein the suitable modeling is influenced by the design of the pump. Thus when using a displacement pump such as a diaphragm pump, a hose roller pump, a piston pump or a geared pump as the dialysis fluid pump, the modeling of the dialysis fluid pump as a current source is possible. The situation is similar when the blood pump is embodied as a displacement pump, for example, as the hose roller pump. A constant pressure pump such as an impeller pump is preferably modeled as a voltage source. If the blood pump or the dialysis fluid pump is modeled as a non-ideal voltage or current source with corresponding internal resistances, then the respective internal resistances must be added to the resistances in the dialysis fluid circulation and/or in the extracorporeal blood circulation. Thus, for example, in modeling the dialysis fluid pump as a non-ideal voltage source, the flow resistance of the dialysis fluid pump must be included in the input resistance 405. The situation is similar for the extracorporeal blood circulation. Those skilled in the art are aware of the corresponding considerations required for this. Those skilled in the art are also aware of how to convert equivalent circuit diagrams in which non-ideal voltage sources are modeled into corresponding equivalent circuit diagrams with non-ideal current sources.

In the dimensioning of the resistances in the extracorporeal blood circulation and in the dialysis fluid circulation as well as in dimensioning the internal resistances of the pumps involved and in controlling the pumps involved to achieve a desired ultrafiltration rate the following considerations may be helpful.

If a corresponding current I_{UF} is assumed in the equivalent circuit diagram for the ultrafiltration rate then in the case of modeling of the pumps as voltage sources, the following formula can be given for the ultrafiltration rate in the case of modeling of the pumps as voltage sources when the dialysis fluid pump is modeled with a voltage source of the voltage U_{D} and the blood pump is modeled with a voltage source of the voltage U_{B}:

$${I}_{UF} = \frac{\frac{{U}_{B} \cdot {R}_{V}}{{R}_{a} + {R}_{V}} - \frac{{U}_{D} \cdot {R}_{out}}{{R}_{in} + {R}_{in}}}{\frac{{R}_{a} \cdot {R}_{V}}{{R}_{a} + {R}_{V}} + \frac{{R}_{in} \cdot {R}_{out}}{{R}_{in} + {R}_{out}} + {R}_{TM}} \qquad ( equation 5 )$$

In the case of modeling of the pumps as current sources, when the dialysis fluid pump is modeled with a current source of the current I_{D} and the blood pump is modeled with a current source of the current I_{B}, the following formulas are given for the ultrafiltration rate:

$${I}_{UF} = \frac{{I}_{B} \cdot {R}_{V} \cdot {I}_{D} {R}_{D}}{{R}_{v} + {R}_{out} + {R}_{TM}} \qquad ( equation 6 )$$

The following consideration is helpful for the dimensioning of the output resistance 404 (symbol Rₒᵤₜ). The rearrangement of equation 6 for the output resistance 404 (symbol Rₒᵤₜ) required to achieve a certain ultrafiltration rate yields the following equation:

$${R}_{out} = \frac{{R}_{y} \cdot ( {I}_{B} - {I}_{UF} ) - {R}_{TM} {I}_{UF}}{{I}_{D} - {I}_{UP}} \qquad ( equation 7 )$$

Equation 7 shows that when the transmembrane resistance R_{TM} is too high, it has an unfavorable influence on the dimensioning of the output resistance Rₒᵤₜ in the dialysis fluid circulation. The transmembrane resistance R_{TM} should therefore be selected to be as low as possible, for example, as a filter with high specific transfer coefficient (“high cut-off filter”) or as a filter with a sufficiently large effective filter area. One factor to be taken into account here is that the transmembrane resistance R_{TM}—like the venous filter longitudinal resistance R_{vF}—will increase in the course of the extracorporeal blood treatment. The increase in the venous filter longitudinal resistance R_{vF} in the extracorporeal blood circulation is typically based on the increase in the hematocrit in the course of the blood treatment, the so-called hemoconcentration, as well as on a possible development of constrictions in the extracorporeal blood circulation. An increase in the transmembrane resistance R_{TM} often occurs in the course of a blood treatment due to deposits on the dialyzer membrane. These effects, which frequently occur in the course of a blood treatment, must be taken into account in the dimensioning of the resistances in the extracorporeal blood circulation and in the dialysis fluid circulation.

The following numerical example can provide a starting point for the possible dimensioning of the flows involved, i.e., a minimal value I_{Bmin}=60 mL/min and a maximal value I_{Bmax}=300 mL/min are assumed for the blood flow I_{B}; a maximal value I_{UFmax}=I_{B}/10, i.e., approx. 20 mL/min and the minimal value I_{UFmin}=0 mL/min are assumed for the ultrafiltration rate I_{UF} and a minimal value I_{Dmin}=I_{B}/3 and a maximal value I_{Dmax}=200 mL/min are assumed at dialysis fluid rate I_{D}.

The blood flow I_{B} is set by the blood pump as the flow through the blood pump 115, while the dialysis fluid rate I_{D} corresponds to the flow through the dialysis fluid pump 107 and the ultrafiltration rate I_{UF} corresponds to the flow through the transmembrane resistance 308.

In general the following equation holds for the dialysis fluid rate ID:

$${I}_{D} = \frac{{I}_{B} \cdot {R}_{V} - {I}_{UF} \cdot ( {R}_{V} + {R}_{out} + {R}_{TM} )}{{R}_{out}} \qquad ( equation 8 )$$

Fundamentally at a given blood flow I_{B} a maximum ultrafiltration is achieved when the dialysis fluid rate I_{D} is minimal.

After rearranging equation 8, the following equation is obtained:

$${I}_{D min} \leq \frac{{R}_{y}}{{R}_{out}} \cdot ( {I}_{B} - {I}_{UF} ) - ( 1 + \frac{{R}_{TM}}{{R}_{out}} ) {I}_{UF} \qquad ( equation 9 )$$

If I_{B}=50 mL/min is used as an alternative numerical value for the minimal blood flow and I_{UF}=5 mL/min is used for the ultrafiltration rate, this yields the following equation which shows the ratios of the resistances R_{V}, Rₒᵤₜ and R_{TM} to one another in one equation:

$$\begin{matrix}20 \leq \frac{{R}_{V}}{{R}_{out}} \cdot ( 50 - 5 ) - ( 1 + \frac{{R}_{TM}}{{R}_{out}} ) 5 \\ or \text{:} & ( equation 10 ) \\ 25 \leq 45 \cdot \frac{{R}_{V}}{{R}_{out}} - 5 \cdot \frac{{R}_{TM}}{{R}_{out}} & ( equation 11 )\end{matrix}$$

Starting from a certain ratio between the transmembrane resistance R_{TM} and the starting resistance in the dialysis fluid circulation, the following equations are obtained for the dimensioning of the output resistance Rₒᵤₜ in the dialysis fluid circulation with respect to the total venous resistance R_{V}, where ascending values are given for the transmembrane resistance R_{TM}, reflecting the aforementioned effects of an increase over the course of treatment.

$$\begin{matrix}{R}_{TM} = {R}_{out} \text{/} 2 & -> & {R}_{out} \leq \frac{90}{55} {R}_{V} \\ {R}_{TM} = {R}_{out} & -> & R \leq \frac{45}{30} {R}_{V} \\ {R}_{TM} = 2 \cdot {R}_{out} & -> & {R}_{out} \leq \frac{45}{35} {R}_{V} \\ {R}_{TM} = 4 \cdot {R}_{out} & -> & {R}_{out} \leq \frac{45}{45} {R}_{V}\end{matrix}$$

This sample calculation shows that the output resistance Rₒᵤₜ must be selected to be lower when the transmembrane resistance R_{TM} is greater. As already mentioned above, for the design of the flow resistances in the dialysis fluid circulation and in extracorporeal blood circulation, it is a disadvantage for the transmembrane resistance R_{TM} to be too high. For example, if it is assumed that the transmembrane resistance R_{TM} may assume as the maximum value four times the output resistance Rₒᵤₜ in the dialysis fluid circulation, then the numerical example given above yields the simple requirement that Rₒᵤₜ must be lower than R_{V}. For the design of the output resistance Rₒᵤₜ in the dialysis fluid circulation and the total venous resistance R_{V} it is sufficient if the total venous resistance R_{V} is to be assumed at the start of the blood treatment because equation 9 will be satisfied sooner with a total venous resistance which increases during the blood treatment.

For the subsequent specification of values for the rate of the dialysis fluid flow I_{D}, the blood flow I_{B} and the ultrafiltration rate I_{UF} as examples, it is assumed that at the start of the treatment the value of the output resistance Rₒᵤₜ corresponds to the total venous resistance R_{V} and that the transmembrane resistance R_{TM} at the start of the treatment corresponds to the output resistance Rₒᵤₜ.

In the case of a blood flow I_{B}=200 mL/min, the maximum ultrafiltration rate I_{UF}=20 mL/min is achieved at a dialysis flow rate of

I_{D}=(200−20) ml/min−2·20 ml/min=140 ml/min

based on equation 9.

The minimum ultrafiltration rate I_{UF}=0 mL corresponds to a dialysis fluid rate of

I_{D}=(200−0) ml/min−2·0 ml/min=200 ml/min.

Both values are within an admissible, acceptable or preferred range of the dialysis fluid rate I_{D}≦200 mL/min.

For the following numerical example, it is assumed that in the remaining course of the blood treatment the transmembrane resistance R_{TM} rises to a four-fold value as a result of the effects described above. Throttling of the dialysis fluid rate to

I_{D}=(200−20) ml/min−5·20 ml/min=80 ml/min

would be necessary to achieve a maximum ultrafiltration rate I_{UF}=20 mL/min.

For the minimum ultrafiltration rate I_{UF}=0 mL, a dialysis fluid rate as follows

I_{D}=(200−0) ml/min−5·0 ml/min=200 ml/min

would still have to be established, same as before,

If it is assumed that due to the effects described above, the total venous resistance R_{V} will double in the course of the blood treatment, then this yields a required dialysis flow rate of

I_{D}=2·(200−20) ml/min−5·20 ml/min=260 ml/min

for a maximum ultrafiltration rate of 20 mL/min to be achieved.

For a minimum ultrafiltration rate I_{UF}=0 mL, a dialysis flow of

I_{D}=2·(2.00−0) ml/min−5·0 ml/min=4 00 ml/min

is obtained.

To be able to regulate the ultrafiltration rate in this case over the entire range of 0 to 20 mL/min, there is the possibility of expanding the range of the dialysis fluid flow which is regarded as admissible, acceptable or preferred or to reduce the blood flow I_{B}. Thus, for example, with a maximum allowed dialysis fluid flow I_{D}=200 mL/min, a maximum ultrafiltration rate I_{UF}=20 mL/min at a blood flow I_{B}=170 mL/min would be achieved, and a minimum ultrafiltration rate I_{UF}=0 mL/min would be achieved at a blood flow I_{B}=100 mL/min.

The following sample calculation should illustrate how advantageous it is to keep the transmembrane resistance R_{TM} low. If one makes sure, for example, through the dimensioning of the dialyzer and of the dialysis fluid circulation that the transmembrane resistance R_{TM} will correspond at most to the output resistance Rₒᵤₜ in the dialysis fluid circulation and if the dialysis fluid circulation and the extracorporeal blood circulation are designed so that the output resistance Rₒᵤₜ in the dialysis fluid circulation is in a ratio of Rₒᵤₜ=3/2 R_{V} to the total venous resistance, then a maximum ultrafiltration rate of 20 mL/min is obtained at a blood flow I_{B}=200 mL/min and a dialysis fluid rate of

I_{D}=4/3·(200−20) ml/min−2·20 ml/min=200 ml/min

and a minimum ultrafiltration rate of 0 mL/min is obtained at the same blood flow I_{B} and a dialysis fluid rate of

I_{D}=4/3·(200−0) ml/min−2·0 ml/min=267 ml/min

The range of an ultrafiltration rate I_{UF} of 0 mL/min to 20 mL/min may thus be controlled with a minor variation in the dialysis fluid rate I_{D}.

1. A device (1, 2) for controlling or regulating an ultrafiltration in a dialysis treatment
in which blood to be ultrafiltered flows in an extracorporeal blood circulation (112) through a blood chamber (110) of a dialyzer (113), which is divided by a semipermeable membrane (111) into the blood chamber (110) and a dialysis fluid chamber (108), and dialysis fluid in a dialysis fluid circulation (109) flows through the dialysis fluid chamber (108) of the dialyzer (113),
having a blood pump (115) for creating a blood flow in the extracorporeal blood circulation (112), having a dialysis fluid pump (107) for creating a dialysis fluid flow in the dialysis fluid circulation (109),
having a balancing device (104) for setting up a fluid balance in the dialysis fluid circulation between an inflow (106) and an outflow (105) of the dialysis fluid chamber (113) as a measure of the ultrafiltration,
a regulating unit (101) for regulating the blood pump (115) and/or the dialysis fluid pump (107) so that a predetermined ultrafiltration is achieved without any further active control or regulation of the dialysis fluid flow flowing into the dialyzer (113) or flowing out of the dialyzer.

2. The device (1, 2) for controlling or regulating an ultrafiltration according to claim 1, wherein the regulating unit (101) is adapted to preselect an ultrafiltration rate and/or an ultrafiltration volume to be withdrawn during the course of a treatment.

3. The device (1, 2) according to claim 1, wherein the dialysis fluid pump (107) is arranged in an inlet line to the dialysis fluid chamber (113) and there is not further active control or regulation of the dialysis fluid flow downstream from the dialysis fluid chamber (113).

4. The device (1, 2) according to claim 1, wherein the blood pump is arranged in an inlet line to the blood chamber.

5. The device (1, 2) according to claim 1, wherein a predetermined value can be adjusted for the blood flow and the regulating unit is adapted for regulating the dialysis fluid flow.

6. The device (1, 2) according to claim 1, wherein a predetermined value can be set for the dialysis fluid flow, and the regulating unit is adapted for regulating the blood flow.

7. The device (1, 2) according to claim 1, wherein a profile for the ultrafiltration rate can be preselected by the regulating unit (101) such that intervals with a positive ultrafiltration rate alternative with intervals with a negative ultrafiltration rate.

8. The device (2) according to claim 1, wherein the balancing device has a differential flow measuring unit (104) for measuring the differential flow between an inflow into the dialysis fluid chamber (108) and the outflow out of the dialysis fluid chamber, a branch from the inflow or the outflow to the branching point of dialysis fluid from the inflow or from the outflow into another flow path (212), as well as a device for adjusting the flow rate (211) in the inflow, in the outflow and/or in the additional flow path, this device being controllable so that, the measured differential flow satisfies a predetermined condition and having a device (211) for determining the flow rate in the additional flow path as a measure of the fluid balance.

9. The device (2) according to claim 7, wherein the predetermined condition of the differential flow is based on an integration of the differential flow over a predetermined integration interval.

10. The device according to claim 1, wherein the blood circulation, a part of the blood circulation, the dialysis fluid circulation or a part of the dialysis fluid circulation is designed as a disposable article.

11. A method for controlling or regulating an ultrafiltration in a dialysis treatment in which blood to be ultrafiltered in an extracorporeal blood circulation (112) flows through a blood chamber (110) of a dialyzer (113) which is divided by a semipermeable membrane (111) into the blood chamber (110) and a dialysis fluid chamber (108), and dialysis fluid in a dialysis fluid circulation (109) flows through the dialysis fluid chamber (108) of the dialyzer (113), and in which a blood pump (115) for creating a blood flow in the extracorporeal blood circulation (112), a dialysis fluid pump (107) for creating a dialysis fluid flow in the dialysis fluid circulation (109) and a balancing device (104) for setting up a fluid balance in the dialysis fluid circulation (109) between an inflow (106) and an outflow (105) of the dialysis fluid chamber (108) as a measure of the ultrafiltration are provided, such that the method comprises the following:
regulating the blood pump (115) and/or the dialysis fluid pump (107), so that a predetermined ultrafiltration is achieved without any additional or further active control or regulation of the dialysis fluid flow flowing out of the dialyzer (113) or into the dialyzer (113).

12. The method for controlling or regulating an ultrafiltration according to claim 11, wherein the balancing between an inflow and an outflow from the dialysis fluid chamber comprises the following:
measuring a differential, flow between an inflow into the dialysis fluid chamber and an outflow out of the dialysis fluid chamber (206),
using the measured differential flow as a manipulated variable for the equipment for setting the flow rate in an additional flow path (212) which branches off from the inflow or the outflow and determining the flow rate in the additional flow path (212) as a measure of the fluid balance.

13. The method for controlling or regulating an ultrafiltration claim 1, wherein a profile for the ultrafiltration rate is preselected in which intervals with a positive ultrafiltration rate alternative with intervals with a negative ultrafiltration rate.

14. The method for controlling or regulating an ultrafiltration according to claim 12, wherein a predetermined condition is satisfied for the differential flow based on integration of the differential flow over a predetermined integration interval.

15. The method for controlling or regulating an ultrafiltration by means of a device (1, 2) according to claim 8, wherein the balancing between an inflow and an outflow from the dialysis fluid chamber comprises the following:
measuring a differential flow between an inflow into the dialysis fluid chamber (205) and an outflow out of the dialysis fluid chamber (206),
using the measured differential flow as a manipulated variable for the equipment for setting the flow rate in an additional flow path (212) which branches off from the inflow or the outflow and determining the flow rate in the additional flow path (212) as a measure of the fluid balance.
